(19) United States
(12) Patent Application Publication
(10) Pub. No.: US 2011/0105970 A1
(43) Pub. Date: May 5, 2011

(54) ANATOMICAL SUPPORT BRACES AND METHODS

(76) Inventor: James V. GAINER, JR., Gainesville, FL (US)

(21) Appl. No.: 12/611,075

(22) Filed: Nov. 2, 2009

(51) Int. Cl. A61F 5/00

(52) U.S. Cl. 602/19

(57) ABSTRACT

Anatomical support braces surround an anatomical area in need of support and provide an inwardly directed support force of substantially uniform magnitude and substantially symmetrical direction. Certain preferred embodiments of the anatomical support braces are provided with a flexible support web having opposed terminal ends and a length sufficient to surround the anatomical area in need of support and to allow portions of the opposed terminal ends to be overlapped with one another. Web fasteners (e.g., a two-part hook and loop fastening system) are associated with the opposed terminal ends for attaching the terminal ends to one another when overlapped to thereby positionally retain the support web in surrounding relationship to the anatomical area in need of support. When attached to one another, the web support will establish sufficient tension such that a supporting force is directed inwardly toward a central region of the anatomical area surrounded by the support web. A viscoelastic memory foam layer is attached to an interior surface of the support web and establishes substantially uniform magnitude and substantially symmetrical direction of the inwardly directed supporting force.

### FIELD

The disclosed subject matter relates generally to devices and methods for providing braced anatomical support and stabilization.

### BACKGROUND

It is often desirable to support various anatomical structures for the purpose of treating orthopedic and/or muscular ailments and the amelioration of pain that such ailments may cause. For example, belt-like braces for a patient's lumbosacral region are well known as evidenced from U.S. Pat. Nos. 1,932,270; 3,717,143; 4,475,543; and 4,572,167 (the entire content of each prior-issued U.S. patent being expressly incorporated hereinto by reference). In general, such conventional braces will include a flexible web support belt sized to encompass the anatomical region in need of support and adapted to be tensioned about the anatomical region to thereby exert inward pressure and thereby provide patient-external orthopedic/muscular support.

Anatomical braces are conventionally provided in many different styles and types to provide protection to various anatomical parts. By way of example, braces are provided conventionally to stabilize movement of joints and bones in cases where excessive movement could have deleterious effects, or simply cause pain. In addition, braces are provided conventionally to stabilize muscle groups and ligaments to reduce pain and aid healing. Temporary braces may also provide rigid support to an extremity or the spine to enable transport to a treatment facility.

In general, the amount of support provided by a brace is directly proportional to the amount of discomfort the brace causes the patient. The amount of support provided by a particular brace is also proportional to the percentage of force vectors which are successfully directed toward the center of the anatomical region that is surrounded by the brace. In order to successfully stabilize an anatomical region, whether central or peripheral, it is necessary to have uniform and symmetrical force vectors (i.e., uniform force magnitude and symmetrical force direction) originating from the peripheral circumference of the stabilizing brace toward the central area of the anatomical region being stabilized.

Because the body surface is very irregular, the force vectors originating in the outer circumference of the brace when it is tensioned can be distorted. Some force vectors are in fact directed toward the intended central area, but many are either redirected or decreased in intensity by a patient's body surface irregularities and/or by minute spaces between the brace and the patient's body. The result of such irregularities is that a significant degradation in the net force and direction applied to the central area occurs. As a result of this force non-uniformity and directional asymmetry, the anatomical region may not in fact be provided with support that is adequate to stabilize the intended anatomical central region and ameliorate pain.

It would therefore be especially desirable to provide anatomical support braces and methods whereby the external support pressure is essentially uniform in terms of the applied force magnitude and essentially symmetrical in terms of the applied force direction. It is towards fulfilling such a need that the present invention is directed.

### SUMMARY OF EXEMPLARY EMBODIMENTS

Broadly, preferred embodiments are provided in the form of anatomical support braces which surround an anatomical area in need of support and provide an inwardly directed support force of substantially uniform magnitude and substantially symmetrical direction.

According to certain preferred embodiments, anatomical support braces are provided with a flexible support web having opposed terminal ends and a length sufficient to surround the anatomical area in need of support and to allow portions of the opposed terminal ends to be overlapped with one another. Web fasteners (e.g., a two-part hook and loop fastening system) are associated with the opposed terminal ends for attaching the terminal ends to one another when overlapped to thereby positionally retain the support web in surrounding relationship to the anatomical area in need of support. When attached to one another, the web support will establish sufficient tension such that a supporting force is directed inwardly toward a central region of the anatomical area surrounded by the support web. A viscoelastic memory foam layer is attached to an interior surface of the support web and establishes substantially uniform magnitude and substantially symmetrical direction of the inwardly directed supporting force.

In some embodiments, the viscoelastic memory foam layer has a length that is sufficient to surround the central region of the anatomical area by an angle θ of at least about 180°, preferably at least about 200°, and most preferably at least about 270°. The viscoelastic memory foam layer could substantially entirely surround the central region of the anatomical area (i.e., the angle θ is substantially about 360°).

The viscoelastic memory foam layer may include a flexible cover defining an interior pocket, and a viscoelastic memory foam member inserted into the pocket of the cover. In certain embodiments, the cover may include an opening to allow insertion and removal of the viscoelastic memory foam member, and cover fasteners for releasably closing the opening (e.g., a two-part hook and loop fastening system). The cover may be removably attached to the web support (e.g., so as to allow replacement and/or interchangeability). For such purpose, a two-part fastening system may be provided to removably attach the cover to the web support. In such embodiments, one part of the two-part fastening system is operatively associated with the cover and another part of the two-part fastening system is operatively associated with the support web. The two-part fastening system may be in the form of a two-part hook and loop fastening system, wherein one part of the fastening system bounds a peripheral region of the cover, and the other part of the fastening system bounds a central interior surface of the support web in opposition to the peripheral region of the cover.

In use, support may be provided to an anatomical area by (a) providing an anatomical support brace having the above-described attributes, (b) wrapping the support web of the brace around the anatomical area in need of support, (c) overlapping the opposed terminal ends of the support web; and (d) fastening the fasteners of the support web to one another so as to establish sufficient tension such that a supporting force of substantially uniform magnitude and substantially symmetrical direction is directed inwardly toward a central region of the anatomical area surrounded by the support web by means of the viscoelastic memory foam layer.

These and other aspects and advantages of the present invention will become more clear after careful consideration is given to the following detailed description of the preferred exemplary embodiments thereof.

### BRIEF DESCRIPTION OF ACCOMPANYING DRAWINGS

The disclosed embodiments of the present invention will be better and more completely understood by referring to the following detailed description of exemplary non-limiting illustrative embodiments in conjunction with the drawings of which:

FIG. 1 is a rear perspective view of a partial human torso showing a lumbosacral brace in accordance with one embodiment of the present invention;

FIG. 2 is an exploded anterior view of the brace shown in FIG. 1;

FIG. 3 is a side elevational view showing the viscoelastic memory foam insert employed in the brace of FIG. 1;

FIG. 4 is a top cross-sectional schematic view showing the force vectors achieved by a conventional lumbosacral brace; and

FIG. 5 is a top cross-sectional schematic view similar to FIG. 4 but showing the force vectors achieved a lumbosacral brace in accordance with an embodiment of the present invention.

### DETAILED DESCRIPTION

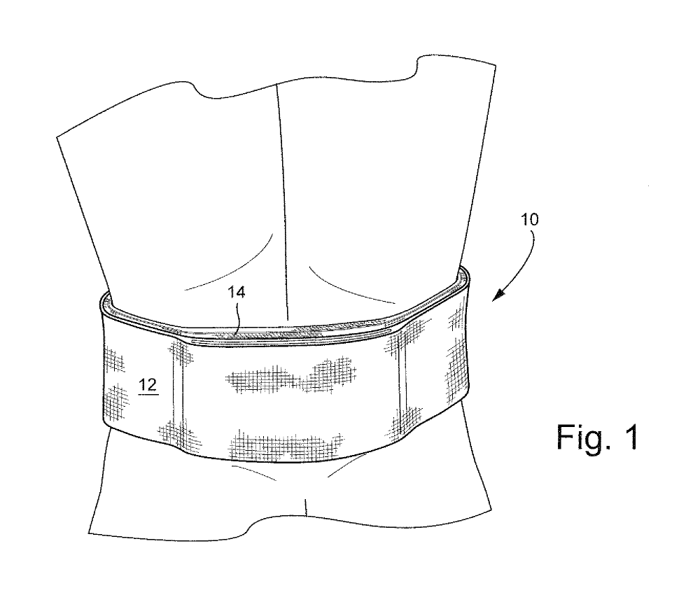

Accompanying FIG. 1 depicts one preferred form of an anatomical support brace 10 in accordance with a presently preferred embodiment of the present invention. In this regard, it will be observed that the brace 10 is in the form of a lumbosacral support device which is sized and configured to support a patient's lumbosacral region. It will be appreciated however that the depiction of the brace 10 in the form of a lumbosacral support device is exemplary only and represents a presently preferred embodiment of the invention. The concepts and attributes of the invention that will be discussed in relation to such presently preferred embodiment could equivalently be embodied in other orthopedic and/or muscular support devices to support virtually any anatomical region, including by way of example only those devices adapted for anatomically supporting a patient's entire spine, arm, leg, hand, knee, elbow, wrist and/or ankle regions. Furthermore, although a human patient is depicted in FIG. 1, the present invention could equivalently be embodied in devices adapted for veterinary anatomical support applications.

The support brace 10 will advantageously include a flexible support web 12 which is sized so as to encompass or wrap the anatomical part of the patient in need of orthopedic support. In preferred forms, the support web 12 is a fabric structure constructed of light-weight synthetic and/or natural filaments. Alternatively, the support web could be formed from leather or leather-like material.

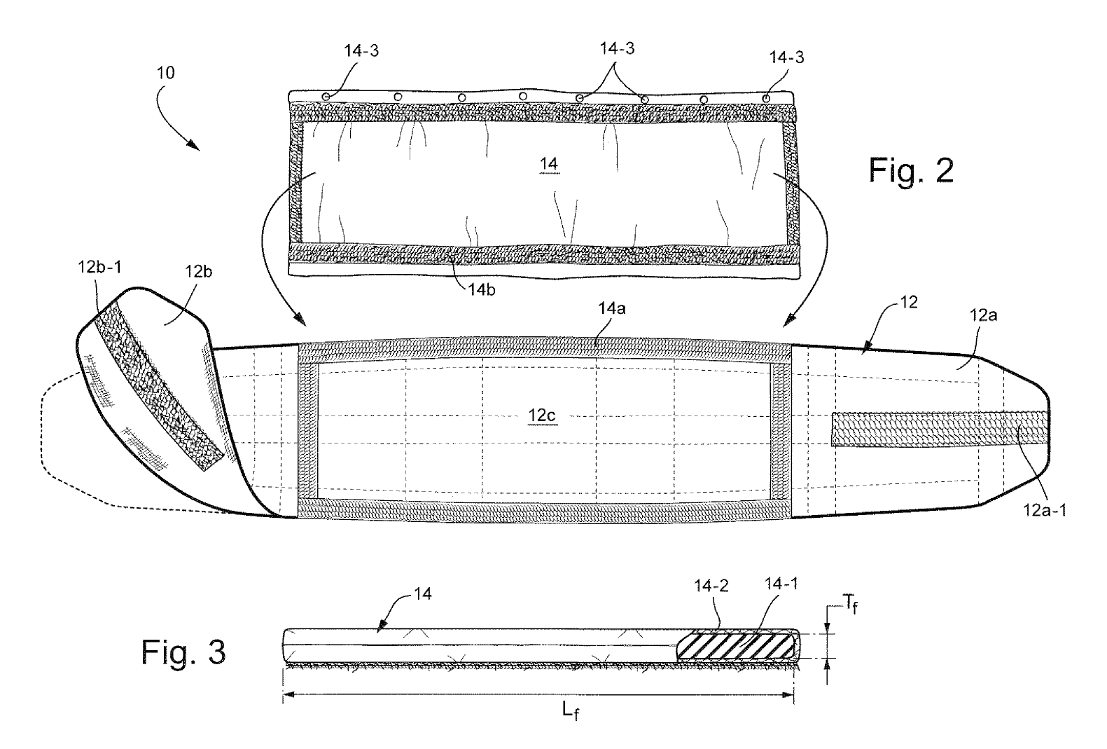

As depicted in FIG. 1, the support web 12 is of sufficient length overall to be wrapped about a patient's torso for supporting the lumbosacral region. The terminal ends of the 12a, 12b of the support web will thus include suitable fasteners 12a-1 and 12b-1, respectively, which will connection the ends to one another when overlapped (see FIG. 2). Preferred embodiments will include two-part VELCRO® brand hook and loop fasteners for such purpose, but other equivalent fastening systems can be employed including without limitation, two-part snaps, quick release buckles, buttons/holes, grommets/laces and the like. Suffice it to say, the fasteners 12a-1 and 12-b1 in whatever form must be capable of securely mating the ends 12a, 12b to one another to maintain the desired tension on the support web 12 which in turn is translated into pressure applied onto the anatomical region which it surrounds.

Important to the present invention, the interior (patient-side) of the support web 12 is provided with a removable support pad 14 which includes a layer (or layers) of viscoelastic memory foam 14-1 inserted into an interior pocket of a covering layer 14-2 (see FIG. 3). The pocket 14-2 may be closed by means of snaps (a representative few of which are depicted in FIG. 3 by reference numeral 14-3) or other suitable equivalent fastening systems as have been mentioned previously.

Virtually any conventional viscoelastic memory foam material may be employed in the practice of the present invention as the layer 14-1. Preferred are viscoelastic polyurethane foams having an Indentation Force Deflection (IFD) measured at 25% according to ASTM D3574 of between about 10 lbs. to about 35 lbs., preferably between about 12 lbs. to about 20 lbs. Advantageously, the IFD measured at 25% will be between about 14 to about 16 lbs. The viscoelastic memory foams will have a falling ball resilience (ASTM D3574) of at most 10%, preferably at most 5% and advantageously at most 2.5%, and a percent recovery after 25% IFD (measured by dividing the height of the foam after being subjected to 25% IFD by the height of the foam during 25% IFD times 100) of at least about 85%, preferably at least about 90% and advantageously at least about 95%. Suitable viscoelastic memory foams may be made by techniques well known in the art, for example as described in U.S. Pat. Nos. 6,946,497; 7,208,531; and 7,238,730 (each being incorporated fully hereinto by reference).

The support pad 14 is removably attached to a central area 12c of the interior (patient-side) surface of the support web 12 so that support pads having different properties and/or physical characteristics (e.g., thickness) may be provided depending on a patient's particular needs. Most preferably, the removable attachment is provided by a two-part VELCRO® brand hook and loop fastener system whereby one part 14a is attached to and bounds the central area 12c of the support web 12 and the other part 14b is attached to and bounds a corresponding opposed peripheral surface region of the support pad covering layer 14-2 so as to be conformably mated with the part 14a.

The particular thickness T_{f} (see FIG. 3) of the viscoelastic memory foam layer (or layers) 14-1 is not critical provided that sufficient support forces are capable of being developed for a patient's particular needs. Thus, the foam layer (or layers) of viscoelastic memory foam 14-1 must have a minimum thickness sufficient to provide adequate stabilization pressures but must not be excessively thick so that it is uncomfortably bulky when mated with the support web 12. In general, the viscoelastic memory foam will typically have a thickness T_{f} of at least about 0.5 inches up to about 2.0 inches, and preferably between about 0.75 inches to about 1.5 inches.

The length L_{f} (see FIG. 3) of the viscoelastic memory foam 14-1 will depend on the particular anatomical region being stabilized by the brace 10. In general, the length L_{f} of the viscoelastic memory foam 14-1 will be such that it surrounds the anatomical region being stabilized through an angle θ (see FIG. 5) of at least about 180°, more preferably at least about 200°, and most preferably at least about 270°. In certain applications, the length L_{f} of the viscoelastic memory foam 14-1 will be such that it will substantially completely surround the anatomical region being stabilized (i.e., angle θ is substantially about 360°).

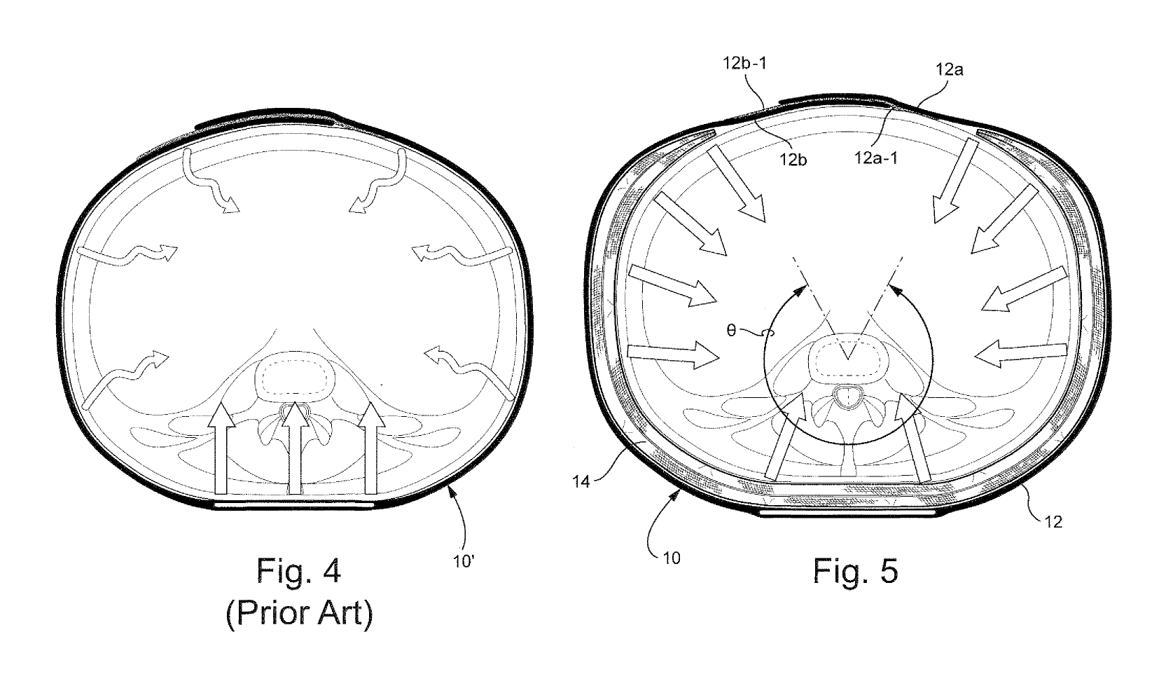

In use, the brace 10 will be positioned around the patient's anatomical region in need of stabilization with the terminal ends 12a, 12b being overlapped and attached to one another to provide sufficient tension on the support web 14. This in turn will create a pressure force directed inwardly of the anatomical region surrounded by the brace 10. As shown, in FIG. 4, a conventional brace 10′ (that is a brace that includes a support web 12 but does not include a support pad 14 as in the present invention) will exhibit a non-uniform force magnitude and asymmetric force direction. However, as shown in FIG. 5, the brace 10 according to the present invention exerts a force that is both uniform in magnitude and directionally symmetric with respect to the anatomical region being stabilized.

A further understanding of the present invention will be obtained from the following non-limiting Example.

### Example

A lumbosacral brace with inserts of viscoelastic polyurethane memory foam was used to treat a patient with low back pain and bilateral sciatica secondary to a lumbar spinal stenosis and associated mobile spondylolisthesis.

The patient was a 73 year old female nurse with a 4-5 month history of progressively increasing low back pain and bilateral leg pain, right greater than left. Her symptoms began spontaneously and consisted of low back pain with radiation to both legs and top of the feet, right greater than left. The symptoms were worse with standing and walking, and were also aggravated with sitting. She had pain relief only with lying down and with narcotic pain medication. Her symptoms were worse in the afternoon and evening and had progressed to the point where she was unable to continue working as a school nurse.

Her history was significant for diabetes requiring diet and oral medication, and hypertension. She was 5 feet tall, and weighed 165 pounds. Her neurologic examination was positive for right L5 dermatome hypalgesia, and mild boot hypalgesia of both feet. There was mild dorsiflexion weakness of the right foot. Tendon reflexes were 2 plus at the knees and absent at the ankles. Laseague's sign was positive on the right. Flexion-extension plain lumbar x-rays showed an anterolisthesis of 8 mm with flexion and complete reduction with extension.

An MRI scan had been done and showed a severe spinal stenosis at L3-4 and L4-5. Following the MRI the patient was seen by a spine surgery team and physical therapy was recommended. The patient stopped physical therapy as it made her symptoms worse. She was then seen by another spine surgeon who recommended an open decompression of L3-5. A second opinion was requested which resulted in a recommendation of minimally invasive decompression and fusion of L3-5.

The patient's pain increased significantly while waiting to have the decompression and fusion of L3-5 accomplished and arranging long distance transportation to the surgery location. The patient had stopped using a QuikDraw™ Pro (Aspen Medical Products Inc.) lumbosacral brace for support which she had earlier been provided and which had not worked to ameliorate her pain. This conventional brace was modified by attaching inserts of viscoelastic polyurethane memory foam. With use of this modified brace in accordance with the present invention the patient's symptoms with standing and walking were considerably improved and her symptoms with sitting were eliminated.

After about 3 weeks, the patient had surgery. Postoperatively the modified brace in accordance with the present invention was very helpful in relieving her muscle pain to an extent that allowed for early discharge from the hospital and an early start for an ambulation and back exercise program. She discontinued use of the brace after 3 weeks since it was no longer needed for support. Within 8 weeks she was symptom free, walking 2 miles daily, driving her car, and able to return to work

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope thereof.

What is claimed is:

1. An anatomical support brace comprising:
a flexible support web having opposed terminal ends and having a length sufficient to surround an anatomical area in need of support and to allow portions of the opposed terminal ends to be overlapped with one another;
web fasteners associated with the opposed terminal ends for attaching the terminal ends to one another when overlapped to thereby positionally retain the support web in surrounding relationship to the anatomical area in need of support and to establish sufficient tension such that a supporting force is directed inwardly toward a central region of the anatomical area surrounded by the support web; and
a viscoelastic memory foam layer attached to an interior surface of the support web for establishing substantially uniform magnitude and substantially symmetrical direction of the inwardly directed supporting force.

2. An anatomical support brace as in claim 1, wherein the viscoelastic memory foam layer has a length that is sufficient to surround the central region of the anatomical area by an angle θ of at least about 180°.

3. An anatomical support brace as in claim 2, wherein angle θ is at least about 200°.

4. An anatomical support brace as in claim 2, wherein angle θ is at least about 270°.

5. An anatomical support brace as in claim 2, wherein angle θ is about 360°.

6. An anatomical support brace as in claim 1, wherein the viscoelastic memory foam layer includes a flexible cover defining an interior pocket, and a viscoelastic memory foam member inserted into the pocket of the cover.

7. An anatomical support brace as in claim 6, wherein the cover includes an opening to allow insertion and removal of the viscoelastic memory foam member, and cover fasteners for releasably closing the opening.

8. An anatomical support brace as in claim 7, wherein at least one of the web fasteners and the cover fasteners includes a two-part hook and loop fastening system.

9. An anatomical support brace as in claim 6, wherein the cover is removably attached to the web support.

10. An anatomical support brace as in claim 9, further comprising a two-part fastening system for removably attaching the cover to the web support.

11. An anatomical support brace as in claim 10, wherein one part of the two-part fastening system is operatively associated with the cover and another part of the two-part fastening system is operatively associated with the support web.

12. An anatomical support brace as in claim 11, wherein the two-part fastening system includes a two-part hook and loop fastening system.

13. An anatomical support brace as in claim 12, wherein the one part of the fastening system bounds a peripheral region of the cover, and the another part of the fastening system bounds a central interior surface of the support web in opposition to the peripheral region of the cover.

14. An anatomical support brace as in claim 1, wherein the viscoelastic memory foam layer has an Indentation Force Deflection (IFD) measured at 25% according to ASTM D3574 of between about 10 lbs. to about 35 lbs.

15. An anatomical support brace as in claim 14, wherein the viscoelastic memory foam layer has a falling ball resilience according to ASTM D3574 of at most 10%.

16. A method to provide support to an anatomical area comprising:
(a) providing an anatomical support brace as in claim 1;
(b) wrapping the support web of the brace around the anatomical area in need of support; and
(c) overlapping the opposed terminal ends of the support web; and
(d) fastening the fasteners of the support web to one another so as to establish sufficient tension such that a supporting force of substantially uniform magnitude and substantially symmetrical direction is directed inwardly toward a central region of the anatomical area surrounded by the support web by means of the viscoelastic memory foam layer.
